(19) United States
(12) Patent Application Publication
(10) Pub. No.: US 2010/0009917 A1
(43) Pub. Date: Jan. 14, 2010

(54) Modulating Enzymatic Processes by Addition of Diolcontaining Substances

(75) Inventors: Jens Buchardt, Copenhagan (DK); Nils Langeland Johansen, Copenhagen (DK); Magali Zundel, Dyssegaard (DK)

Correspondence Address: NOVO NORDISK, INC.;INTELLECTUAL PROPERTY DEPARTMENT, 100 COLLEGE ROAD WEST, PRINCETON, NJ, 08540, US

(73) Assignee: Novo Nordisk Health Care AG, Zurich (CH)

(21) Appl. No.: 12/526,659

(22) PCT Filed: Feb. 20, 2008
(86) PCT No.: PCT/EP08/52069

§ 371 Date: Sep. 23, 2009

(30) Foreign Application Priority Data
Feb. 22, 2007 (EP) 07102883.1

(51) Int. Cl. A61K 38/16; C12P 21/06; C07K 14/00

(52) U.S. Cl. 514/12; 435/68.1; 530/350

(57) ABSTRACT

The present invention relates to a method for increasing the selectivity of an enzyme, which method comprises performing the reaction in the presence of an enhancing compound selected from one of ethylene glycol, propylene glycol or glycerol.

### FIELD OF THE INVENTION

The present invention relates to a method for increasing the selectivity of an enzyme.

### BACKGROUND OF THE INVENTION

Enzyme activity can be affected by other molecules, for example inhibitors decrease enzyme activity, whilst activators increase enzyme activity. Furthermore molecules may also increase the selectivity of an enzyme.

WO 2005/070468 discloses the use of transglutaminase for catalysing the incorporation of a compound comprising a suitable functional group into a peptide, where said functional group is subsequently used as a further point to conjugate. However the main product could not be obtained at a yield higher than 50%. Thus there is a need for a method to improve the selectivity of the enzyme to increase the economy of the reaction.

### SUMMARY OF THE INVENTION

In one aspect of the invention, there is provided a method for enhancing the selectivity and/or yield of an enzyme-catalysed reaction, said method comprising performing the reaction in the presence of an enhancing compound selected from one of ethylene glycol, propylene glycol or glycerol.

In one aspect of the invention, the enzyme-catalysed reaction comprises covalent bond formation between a first compound and a peptide, and said method comprises reacting in one or more steps the first compound with the peptide in the presence of the enzyme and said enhancing compound.

In one aspect of the invention, there is also provided a peptide obtainable by the method.

### BRIEF DESCRIPTION OF THE FIGURES

FIG. 1 illustrates the reaction profiles for the enzyme reaction in the presence and in the absence of ethylene glycol.

FIG. 2 shows the electropherograms obtained from the transamination of hGH in the presence and in the absence of ethylene glycol.

FIG. 3 illustrates the reaction profiles of TGase Activa WM and TGase MTG P2 in the presence and in the absence of ethylene glycol.

FIG. 4 illustrates the reaction profiles for the enzyme reaction in the presence of 10 to 40% v/v ethylene glycol.

FIG. 5 illustrates the reaction profiles for the enzyme reaction in the presence of 40 to 70% v/v ethylene glycol.

FIG. 6 illustrates the reaction profiles of differing concentrations of TGase MTG P2 in the presence and in the absence of ethylene glycol.

FIG. 7 illustrates the reaction profiles for the enzyme reaction in the presence of ethylene glycol, glycerol, propylene glycol or 1,3 propanediol.

FIG. 8 illustrates the reaction profiles of differing concentrations of TGase MTG P2 in the presence of glycerol.

### DETAILED DESCRIPTION OF THE INVENTION

“Transglutaminase” (E.C.2.3.2.13) is also known as protein-glutamine-γ-glutamyltransferase and catalyses the general reaction:

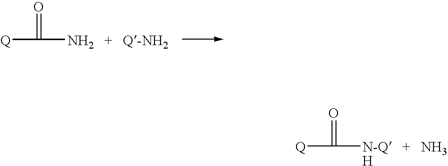

The amine acceptor may represent a glutamine containing peptide and the amine donor then represents a first compound. Alternatively the amine acceptor may represent a first compound and the amine donor then represents a lysine containing peptide.

Different transglutaminases differ from each other, e.g. in what amino acid residues around the Gln are required for the peptide to be a substrate, i.e. different transglutaminases will have different Gln-containing peptides as substrates depending on what amino acid residues are neighbours to the Gln residue.

Examples of useful transglutaminases include microbial transglutaminases, such as e.g. from Streptomyces mobaraense, Streptomyces cinnamoneum and Streptomyces griseocarneum (all disclosed in U.S. Pat. No. 5,156,956, which is incorporated herein by reference), and Streptomyces lavendulae (disclosed in U.S. Pat. No. 5,252,469, which is incorporated herein by reference) and Streptomyces ladakanum (JP2003199569, which is incorporated herein by reference). It should be noted that members of the former genus Streptoverticillium are now included in the genus Streptomyces (Kaempfer, J. Gen. Microbiol. 137, 1831-1892 (1991)). Other useful microbial transglutaminases have been isolated from Bacillus subtilis (disclosed in U.S. Pat. No. 5,731,183, which is incorporated herein by reference) and from various Myxomycetes. Other examples of useful microbial transglutaminases are those disclosed in WO 96/06931 (e.g. transglutaminase from Bacillus lydicus) and WO 96/22366, both of which are incorporated herein by reference. Useful non-microbial transglutaminases include guinea pig liver transglutaminase, and transglutaminases from various marine sources like the flat fish Pagrus major (disclosed in EP 0555649, which is incorporated herein by reference), and the Japanese oyster Crassostrea gigas (disclosed in U.S. Pat. No. 5,736,356, which is incorporated herein by reference).

The invention is based on the finding that certain molecules enhance the selectivity of an enzyme. In one aspect of the invention, there is provided a method for enhancing the selectivity and/or yield of an enzyme-catalysed reaction, said method comprising performing the reaction in the presence of an enhancing compound selected from one of ethylene glycol, propylene glycol or glycerol. In one embodiment, the enhancing compound is ethylene glycol.

Addition of the compound advantageously helps to improve the economy of the reaction. Further, the enhancing compound makes the reaction mixture less likely to spoil during storage, as it prevents freezing and precipitation; and it also facilitates easier purification.

In one embodiment, the enhancing compound is present in a concentration range between 20 to 95%. In one embodiment, the enhancing compound is present in a concentration range between 30 to 70%.

It will be appreciated that the concentration of the enhancing compound will also be dependent on the concentration of the enzyme. Optimising the amount of enzyme and the amount of the enhancing compound is all within the ordinary skills of the person skilled in the art, and may be done, for example, using a factorial design.

In one embodiment, the enzyme-catalysed reaction comprises covalent bond formation between a first compound and a peptide, and said method comprises reacting in one or more steps the first compound with the peptide in the presence of the enzyme and the enhancing compound.

In the present context, the words “peptide” and “protein” are used interchangeably and are intended to indicate the same.

The term “peptide” is intended to indicate a compound with two or more amino acid residues linked by a peptide bond. The constituent amino acids may be from the group of the amino acids encoded by the genetic code and they may be natural amino acids which are not encoded by the genetic code, as well as synthetic amino acids. Natural amino acids which are not encoded by the genetic code are e.g. hydroxyproline, γ-carboxyglutamate, ornithine, phosphoserine, D-alanine and D-glutamine. Synthetic amino acids comprise amino acids manufactured by chemical synthesis, i.e. D-isomers of the amino acids encoded by the genetic code such as D-alanine and D-leucine, Aib (a-aminoisobutyric acid), Abu a-aminobutyric acid), Tle (tert-butylglycine), β-alanine, 3-aminomethyl benzoic acid, and anthranilic acid.

The term peptide is also intended to include said compounds substituted with other peptides, saccharides, lipids, or other organic compound, as well as compounds wherein one or more amino acid residue have been chemically modified. The term is also intended to include peptides to which prosthetic groups are attached.

In one embodiment, the enzyme is selected from transglutaminase, sortase, protease, ligase, glycosyltransferase, glycosynthase, glycopeptidase, glycosidase, tyrosinase, lysyloxidase. In one embodiment, the enzyme is transglutaminase.

The peptide has to be a substrate for an enzyme according to the methods of the present invention. For example, if the enzyme is transglutaminase, it is thus a requirement that the peptide contains a Gln or a Lys residue, and in particular a Gln residue.

It is recognised that whether or not a compound is substrate for a given enzyme in principle depends on the reaction conditions, e.g. the time frame. Given sufficient time, many compounds not normally regarded as substrates are, in fact, substrates. When it is stated above that for a given enzyme some residues may be substrates while others are not it is intended to indicate that “others are not” to an extent where the desired selectivity can still be achieved. If one or more residues are substrates for an enzyme, but only when in contact with the enzyme for an extended period of time, and are desired to be left unconjugated, selectivity may be achieved by removing or inactivating the enzyme after a suitable time.

If a given peptide is not a substrate for an enzyme, it is possible to insert one or more residues to make the peptide a substrate. For example, Gln or Lys residues, and in particular Gln residues may be added to the peptide sequence to make the peptide a substrate for transglutaminase. In principle, such may be inserted at any position in the sequence, however, it is preferably inserted at a position where the physiological, such as the therapeutic activity of the peptide is not affected to a degree where the peptide is not useful anymore, e.g. in a therapeutic intervention. Insertions of amino acid residues in peptides can be brought about by standard techniques known to persons skilled in the art, such as post-translational chemical modification or transgenetic techniques.

Any peptide which are substrates to the enzymes as described hereinbefore can be conjugated by the methods of the present invention, for example the peptide may be selected from, but not limited to, enzymes, peptide hormones, growth factors, antibodies, cytokines, receptors, lymphokines and vaccine antigenes, and particular mentioning is made of therapeutic peptides, such as insulin, glucagon like-peptide 1 (GLP-1), glucagon like-peptide 2 (GLP-2), growth hormone, cytokines, trefoil factor peptides (TFF), melanocortin receptor modifiers and factor VII compounds.

The term “conjugate” as a noun is intended to indicate a modified peptide, i.e. a peptide with a moiety bonded to it to modify the properties of said peptide. As a verb, the term is intended to indicate the process of bonding a moiety to a peptide to modify the properties of said peptide.

WO2005/070468, which is incorporated herein by reference, discloses a number of peptides that are particularly applicable in the methods provided by the present invention.

Other classes of peptides or proteins which are applicable in the methods of the present invention include enzymes. Many enzymes are used for various industrial purposes, and particular mentioning is made of hydrolases (proteases, lipases, cellulases, esterases), oxidoreductases (laccases, peroxidases, catalases, superoxide dismutases, lipoxygenases), transferases and isomerases.

Other peptides or proteins applicable in the methods of the present invention include ACTH, corticotropin-releasing factor, angiotensin, calcitonin, insulin and fragments and analogues thereof, glucagon, IGF-1, IGF-2, enterogastrin, gastrin, tetragastrin, pentagastrin, urogastrin, epidermal growth factor, secretin, nerve growth factor, thyrotropin releasing hormone, somatostatin, growth hormone releasing hormone, somatomedin, parathyroid hormone, thrombopoietin, erythropoietin, hypothalamic releasing factors, prolactin, thyroid stimulating hormones, endorphins, enkephalins, vasopressin, oxytocin, opoids and analogues thereof, asparaginase, arginase, arginine deaminase, adenosine deaminase and ribonuclease.

The term “analogue” as used herein referring to a polypeptide means a modified peptide wherein one or more amino acid residues of the peptide have been substituted by other amino acid residues and/or wherein one or more amino acid residues have been deleted from the peptide and or wherein one or more amino acid residues have been added to the peptide. Such addition or deletion of amino acid residues can take place at the N-terminal of the peptide and/or at the C-terminal of the peptide or internally in the sequence. All amino acids for which the optical isomer is not stated are to be understood to mean the L-isomer.

Such peptides may either be isolated from natural sources (e.g. plants, animals or micro-organisms, such as yeast, bacteria, fungi or vira) or they may be synthesised. Peptides from natural sources also include peptides from transgenic sources, e.g. sources which have been genetically modified to express or to increase the expression of a peptide, wherein said peptide may be “natural” in the sense that it exists in nature or “unnatural” in the sense that it only exists due to human intervention.

In one embodiment, the first compound comprises one or more functional groups or latent functional groups that are not accessible in any of the amino acid residues constituting said peptide.

In some embodiments, the first compound is represented by the formula:

H₂N-D-R—X

wherein
D represents a bond or oxygen;
R represents a linker or a bond;
X comprises one or more functional groups or latent functional groups that are not accessible in any of the amino acid residues constituting said peptide.

In such embodiments, X may be selected from or may be activated to keto, aldehyde, —NH—NH₂, —O—C(O)—NH—NH₂, —NH—C(O)—NH—NH₂, —NH—C(S)—NH—NH₂, —NHC(O)—NH—NH—C(O)—NH—NH₂, —NH—NH—C(O)—NH—NH₂, —NH—NH—C(S)—NH—NH₂, —NH—C(O)—C₆H₄—NH—NH₂, —C(O)—NH—NH₂, —O—NH₂, —C(O)—O—NH₂, —NH—C(O)—O—NH₂, —NH—C(S)—O—NH₂, C(O)—NH₂, Ar—NH₂, alkyne, azide or nitril-oxide.

In one embodiment, the first compound is selected from 4-(aminomethyl)phenyl ethanone, 4-(2-aminoethyl)phenyl ethanone, N-(4-acetylphenyl) 2-aminoacetamide, 1-[4-(2-aminoethoxy)phenyl]ethanone, 1-[3-(2-aminoethoxy)phenyl]ethanone, 1,4-bis(aminoxy)butane, 3-oxapentane-1,5-dioxyamine, 1,8-diaminoxy-3,6-dioxaoctane, 1,3-bis(aminoxy)propan-2-ol, 1,11-bis(aminoxy)-3,6,9-trioxaundecane, 1,3-diamino-2-propanol, 1,2-bis(aminoxy)ethane, and 1,3-bis(aminoxy)propane.

In one embodiment, the reaction is buffered to a pH between 6 and 9. In one embodiment, the reaction is buffered to a pH between 7 and 8.6. In one embodiment, the reaction is carried out at a temperature between 15 and 45° C. In one embodiment, the reaction is carried out at a temperature between 20 and 37° C.

In one embodiment, the method additionally comprises the steps of:
(b) optionally activating the latent functional group; and
(c) reacting in one or more steps said functionalised peptide with a second compound comprising one or more functional groups, wherein said functional group(s) do not react with functional groups accessible in the amino acid residues constituting said peptide, and wherein said functional group(s) in said second compound is capable of reacting with said functional group(s) in said first compound so that a covalent bond between said functionalised peptide and second compound is formed.

In one embodiment, the second compound is represented by the formula

Y-E-Z

wherein Y represents a radical comprising one or more functional groups which react with functional groups present in X, and which functional groups do not react with functional groups accessible in said peptide;
E represents a linker or a bond;
Z is the moiety to be conjugated to the peptide.

The reaction between the functional groups comprised in X and Y forms a moiety. The moiety may in principle be of any kind depending on what properties of the final conjugated peptide is desired. In some situation it may be desirable to have a labile bond which can be cleaved at some later stage, e.g. by some enzymatic action or by photolysis. In other situations, it may be desirable to have a stable bond, so that a stable conjugated peptide is obtained. Particular mentioning is made of the type of moieties formed by reactions between amine derivatives and carbonyl groups, such as oxime, hydrazone, phenylhydrazone and semicarbazone moieties.

In the present context, the term “oxime moiety” indicates a moiety comprising a “oxime bond”, which is intended to indicate a moiety of the formula —C═N—O—.

In the present context, the term “hydrazone moiety” indicates a moiety comprising a “hydrazone bond”, which is intended to indicate a moiety of the formula —C═N—N—.

In the present context, the term “phenylhydrazone moiety” indicates a moiety comprising a “phenylhydrazone bond”, which is intended to indicate a moiety of the formula:

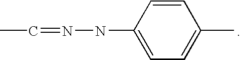

In the present context, the term “semicarbazone moiety” indicates a moiety comprising a “semicarbazone bond”, which is intended to indicate a moiety of the formula —C═N—N—C(O)—N—.

In one embodiment the functional groups of X and Y are selected from amongst carbonyl groups, such as keto and aldehyde groups, and amino derivatives.

It is to be understood, that if the functional group comprised in X is a carbonyl group, then the functional group comprised in Y is an amine derivative, and vice versa. Due to the presence of —NH₂ groups in most peptides, a better selectivity may be obtained if X comprises a keto- or an aldehyde-functionality.

Another example of a suitable pair of functional groups present in X and Y is azide derivatives (—N₃) and alkynes which react to form a triazole moiety. Still another example of a suitable pair is alkyne and nitril-oxide which react to form a isooxazolidine moiety.

It is to be understood that the functional group comprised in X may be latent in the sense that it has to be activated prior to the reaction with Y-E-Z. By way of example, X may comprise a moiety which upon reaction with a suitable reagent is transformed to an aldehyde or a ketone.

Both the first compound and the second compound comprises a linker, R and E, respectively. These linkers, which are independent of each other, may be absent or selected from amongst alkane, alkene or alkyne diradicals and heteroalkane, heteroalkene and heteroalkyne diradicals, wherein one or more optionally substituted aromatic homocyclic biradical or biradical of a heterocyclic compound, e.g. phenylene or piperidine biradical may be inserted into the aforementioned biradicals. It is to be understood that said linkers may also comprise substitutions by groups selected from amongst hydroxyl, halogen, nitro, cyano, carboxyl, aryl, alkyl and heteroaryl.

The term “alkane” is intended to indicate a saturated, linear, branched and/or cyclic hydrocarbon. Unless specified with another number of carbon atoms, the term is intended to indicate hydrocarbons with from 1 to 30 (both included) carbon atoms, such as 1 to 20 (both included), such as from 1 to 10 (both included), e.g. from 1 to 5 (both included); or from 15 to 30 carbon atoms (both included).

The term “alkene” is intended to indicate a linear, branched and/or cyclic hydrocarbon comprising at least one carbon-carbon double bond. Unless specified with another number of carbon atoms, the term is intended to indicate hydrocarbons with from 2 to 30 (both included) carbon atoms, such as 2 to 20 (both included), such as from 2 to 10 (both included), e.g. from 2 to 5 (both included); or from 15 to 30 carbon atoms (both included).

The term “alkyne” is intended to indicate a linear, branched and/or cyclic hydrocarbon comprising at least one carbon-carbon triple bond, and it may optionally comprise one or more carbon-carbon double bonds. Unless specified with another number of carbon atoms, the term is intended to indicate hydrocarbons with from 2 to 30 (both included) carbon atoms, such as from 2 to 20 (both included), such as from 2 to 10 (both included), e.g. from 2 to 5 (both included); or from 15 to 30 carbon atoms (both included).

The terms “heteroalkane”, “heteroalkene” and “heteroalkyne” is intended to indicate alkanes, alkenes and alkynes as defined above, in which one or more heteroatom or group have been inserted into the structure of said moieties. Examples of hetero groups and atoms include —O—, —S—, —S(O)—, —S(O)₂—, —C(O)— —C(S)— and —N(R*)—, wherein R* represents hydrogen or C1-C6-alkyl.

The term “homocyclic aromatic compound” (or radical) is intended to indicate aromatic hydrocarbons, such as benzene and naphthalene.

The term “heterocyclic compound” is intended to indicate a cyclic compound comprising 5, 6 or 7 ring atoms from which 1, 2, 3 or 4 are heteroatoms selected from N, O and/or S. Examples of heterocyclic aromatic compounds include thiophene, furan, pyran, pyrrole, imidazole, pyrazole, isothiazole, isooxazole, pyridine, pyrazine, pyrimidine, pyridazine, as well as their partly or fully hydrogenated equivalents, such as piperidine, pyrazolidine, pyrrolidine, pyrroline, imidazolidine, imidazoline, piperazine and morpholine.

The term “halogen” is intended to indicate members of the seventh main group of the periodic table, e.g. F, Cl, Br and I.

In the present context, the term “aryl” is intended to indicate a homocyclic aromatic ring radical or a fused homocyclic ring system radical wherein at least one of the rings is aromatic. Typical aryl groups include phenyl, biphenylyl, naphthyl, tetralinyl and the like.

The term “radical” or “biradical” is intended to indicate a compound from which one or two, respectively, hydrogen atoms have been removed. When specifically stated, a radical may also indicate the moiety formed by the formal removal of a larger group of atoms, e.g. hydroxyl, from a compound.

The term “heteroaryl”, as used herein, alone or in combination, refers to an aromatic ring radical with for instance 5 to 7 ring atoms, or to a fused aromatic ring system radical with for instance from 7 to 18 ring atoms, wherein at least on ring is aromatic and contains one or more heteroatoms as ring atoms selected from nitrogen, oxygen, or sulfur heteroatoms, wherein N-oxides and sulfur monoxides and sulfur dioxides are permissible heteroaromatic substitutions. Examples include furanyl, thienyl, thiophenyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, quinolinyl, isoquinolinyl, benzofuranyl, benzothiophenyl, indolyl, and indazolyl, and the like.

Both E and R represent bonds or linkers, and in the present context the term “linker” is intended to indicate a moiety functioning as a means to separate Y from Z and X from NH₂-D-, respectively. One function of the linkers E and R may be to provide adequate flexibility in the linkage between the peptide and the conjugated moiety Z. Typical examples of E and R include straight, branched and/or cyclic C₁₋₁₀alkylene, C₂₋₁₀alkenylene, C₂₋₁₀alkynylene, C₂₋₁₀heteroalkylene, C₂₋₁₀heteroalkenylene, C₂₋₁₀heteroalkynylene, wherein one or more homocyclic aromatic compound biradical or heterocyclic compound biradical may be inserted.

WO 2005/070468, which is incorporated herein by reference, discloses a number of bonds and linkers that are particularly applicable in the methods provided by the present invention.

Particular examples of Z include radicals comprising one or more labels, such as fluorescent markers, such as fluorescein radical, rhodamine radical, Texas Red® radical and phycobili protein radical; enzyme substrates, such as p-nitrophenol acetate radical; and radioactive isotopes, such as Cu-64, Ga67, Ga-68, Zr-89, Ru-97, Tc-99, Rh-105, Pd-109, In-111, I-123, I-125, I-131, Re-186, Re-188, Au-198, Pb-203, At-211, Pb-212 and Bi-212; organic moieties, such as PEG or mPEG radicals and amino derivatives thereof (including straight and branched PEG and mPEG radicals); straight, branched and/or cyclic C₁₋₂₂alkyl, C₂₋₂₂alkenyl, C₂₋₂₂alkynyl, C₁₋₂₂heteroalkyl, C₂₋₂₂heteroalkenyl, C₂₋₂₂heteroalkynyl, wherein one or more homocyclic aromatic compound biradical or heterocyclic compound biradical may be inserted, and wherein said C₁₋₂₂ or C₂₋₂₂ radicals may optionally be substituted with one or more substituents selected from hydroxyl, halogen, carboxyl, heteroaryl and aryl, wherein said aryl or heteroaryl may optionally be further substituted by one or more substituents selected from hydroxyl, halogen, and carboxyl; steroid radicals; lipid radicals; polysaccharide radicals, e.g. dextrans; polyamide radicals e.g. polyamino acid radicals; PVP radicals; PVA radicals; poly(1-3-dioxalane); poly(1,3,6-trioxane); ethylene/maleic anhydride polymer; Cibacron dye stuffs, such as Cibacron Blue 3GA; polyamide chains of specified length, as disclosed in WO 00/12587, which is incorporated herein by reference; and hydroxyalkyl starch, such as e.g. hydroxyethyl starch, such as disclosed in WO 03/074087 and WO 02/80979, both of which are incorporated herein by reference.

The term “PEG” is intended to indicate polyethylene glycol of a molecular weight between approximately 100 and approximately 1,000,000 Da, including analogues thereof, wherein for instance the terminal OH-group has been replaced by an alkoxy group, such as e.g. a methoxy group, an ethoxy group or a propoxy group. In particular, the PEG wherein the terminal —OH group has been replaced by methoxy is referred to as mPEG.

The term “mPEG” (or more properly “mPEGyl”) means a polydisperse or monodisperse radical of the structure

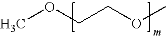

wherein m is an integer larger than 1.

Thus, an mPEG wherein m is 90 has a molecular weight of 3991 Da, i.e. approx 4 kDa. Likewise, an mPEG with an average molecular weight of 20 kDa has an average value for m of 454. Due to the process for producing mPEG these molecules often have a distribution of molecular weights. This distribution is described by the polydispersity index.

The term “polydispersity index” as used herein means the ratio between the weight average molecular weight and the number average molecular weight, as known in the art of polymer chemistry (see e.g. “Polymer Synthesis and Characterization”, J. A. Nairn, University of Utah, 2003).

For example, when reference is made to 20 kDa PEG or 20 kDa mPEG, it is intended to indicate a compound (or in fact a mixture of compounds) with a polydispersity index below 1.06, such as below 1.05, such as below 1.04, such as below 1.03, such as between 1.02 and 1.03.

Particular mentioning is made of C₁₀₋₂₀alkyl, such as C₁₅alkyl and C₁₇alkyl, and in particular linear C₁₅alkyl and C₁₇alkyl, and benzophenone derivatives of the formula:

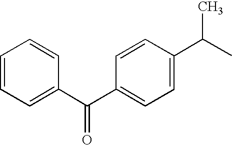

Particular mentioning is made of Z comprising a cibacronyl radical as sketched below:

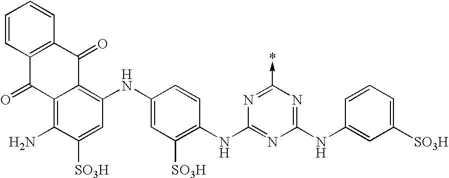

The PEG or mPEG conjugated to a peptide according to the present invention may be of any molecular weight. In particular the molecular weight may be between 500 and 1000,000 Da, such as between 500 and 500,000 Da, such as between 500 and 100,000 Da, such as between 500 and 60,000 Da, such as between 1000 and 40,000 Da, such as between 5000 and 40,000 Da. In particular, PEG with molecular weights of between 10,000 Da and 40,000 Da, such as between 20,000 Da and 40,000 Da, such as between 20,000 and 30,000 Da or between 30,000 and 40,000 Da may be used. Particular mentioning is made of PEG or mPEG with a molecular weight of 10,000, 20,000, 30,000 or 40,000 Da.

Z may be branched so that Z comprises more than one of the above mentioned labels or radicals. For instance, mPEG40K is typically achieved as a branched mPEG with two arm each comprising a mPEG20k.

In one embodiment, Z comprises one or more moieties that are known to bind to plasma proteins, such as e.g. albumin. The ability of a compound to bind to albumin may be determined as described in J. Med. Chem, 43, 1986-1992 (2000), which is incorporated herein by reference. In the present context, a compound is defined as binding to albumin if Ru/Da is above 0.05, such as above 0.10, such as above 0.12 or even above 0.15.

In one embodiment of the invention the albumin binding moiety is a peptide, such as a peptide comprising less than 40 amino acid residues. A number of small peptides which are albumin binding moieties are disclosed in J. Biol Chem. 277(38), 35035-35043 (2002), which is incorporated herein by reference.

WO2005/070468, which is incorporated herein by reference, discloses a number of compounds of the formula Y-E-Z that are particularly applicable in the methods provided by the present invention.

Following the reaction, the peptide may be isolated and purified by techniques well-known in the art. Thus in one embodiment, the invention provides a peptide obtainable according to the methods of the present invention. The peptide may also be converted into a pharmaceutically acceptable salt or prodrug, if relevant. In particular, said method may also comprise a step wherein the resulting conjugated peptide is formulated as a pharmaceutical composition.

In the present context, the term “pharmaceutically acceptable salt” is intended to indicate salts which are not harmful to the patient. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cin-namic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

The term “prodrug” as used herein is intended to indicate a compound which does not necessarily have a therapeutic activity but which upon administration is transformed into a therapeutically active compound by a reaction taking place in the body. Typically such reactions include hydrolysis, e.g. by esterases or oxidations. Examples of prodrugs include biohydrolyzable amides and biohydrolyzable esters and also encompasses a) compounds in which the biohydrolyzable functionality in such a prodrug is encompassed in the compound according to the present invention, and b) compounds which may be oxidized or reduced biologically at a given functional group to yield drug substances according to the present invention. Examples of these functional groups include 1,4-dihydropyridine, N-alkylcarbonyl-1,4-dihydropyridine, 1,4-cyclohexadiene, tert-butyl, and the like.

As used herein, the term “biohydrolyzable ester” is an ester of a drug substance (in casu, a compound according to the invention) which either:
a) does not interfere with the biological activity of the parent substance but confers on that substance advantageous properties in vivo such as duration of action, onset of action, and the like; or
b) is biologically inactive but is readily converted in vivo by the subject to the biologically active principle.

The advantage is, for example increased solubility or that the biohydrolyzable ester is orally absorbed from the gut and is transformed to a compound according to the present invention in plasma. Many examples of such are known in the art and include by way of example lower alkyl esters (e.g., C1-C4), lower acyloxyalkyl esters, lower alkoxyacyloxyalkyl esters, alkoxyacyloxy esters, alkyl acylamino alkyl esters, and choline esters.

As used herein, the term “biohydrolyzable amide” is an amide of a drug substance (in casu, a compound according to the present invention) which either:
a) does not interfere with the biological activity of the parent substance but confers on that substance advantageous properties in vivo such as duration of action, onset of action, and the like; or
b) is biologically inactive but is readily converted in vivo by the subject to the biologically active principle.

The advantage is, for example increased solubility or that the biohydrolyzable amide is orally absorbed from the gut and is transformed to a compound according to the present invention in plasma.

The therapeutic peptides conjugated according to the methods of the present invention may be used in therapy, and this is also an embodiment of the present invention. In one embodiment, the present invention provides the use of conjugated peptides of the present invention in diagnostics.

Insulin is used to treat or prevent diabetes, and in one embodiment, the present invention thus provides a method of treating type 1 or type 2 diabetes, the method comprising administering to a subject in need thereof a therapeutically effective amount of an insulin or insulin compound conjugate according to the present invention. The term “insulin compound” comprises analogues and derivatives of insulin with insulin activity.

GLP-1 may be used in the treatment of hyperglycemia, type 2 diabetes, impaired glucose tolerance, type 1 diabetes, obesity, hypertension, syndrome X, dyslipidemia, β-cell apoptosis, β-cell deficiency, inflammatory bowel syndrome, dyspepsia, cognitive disorders, e.g. cognitive enhancing, neuroprotection, atherosclerosis, coronary heart disease and other cardiovascular disorders.

The term “treatment” and “treating” as used herein means the management and care of a patient for the purpose of combating a condition, such as a disease or a disorder. The term is intended to include the full spectrum of treatments for a given condition from which the patient is suffering, such as administration of the active compound to alleviate the symptoms or complications, to delay the progression of the disease, disorder or condition, to alleviate or relief the symptoms and complications, and/or to cure or eliminate the disease, disorder or condition. The patient to be treated may be a mammal, in particular a human being, but it may also include animals, such as dogs, cats, cows, sheep and pigs.

In one embodiment, the present invention thus provides a method of treating said diseases, the method comprising administering to a subject in need thereof a therapeutically effective amount of a GLP-1 or GLP-1 compound conjugate according to the present invention. The term “GLP-1 compound” comprises analogues and derivatives of GLP-1 with GLP-1 activity.

GLP-2 may be used in the treatment of intestinal failure leading to malabsorption of nutrients in the intestines, and in particular GLP-2 may be used in the treatment of small bowel syndrome, Inflammatory bowel syndrome, Crohn's disease, colitis including collagen colitis, radiation colitis, post radiation atrophy, non-tropical (gluten intolerance) and tropical sprue, damaged tissue after vascular obstruction or trauma, tourist diarrhoea, dehydration, bacteremia, sepsis, anorexia nervosa, damaged tissue after chemotherapy, premature infants, schleroderma, gastritis including atrophic gastritis, postantrectomy atrophic gastritis and helicobacter pylori gastritis, ulcers, enteritis, cul-de-sac, lymphatic obstruction, vascular disease and graft-versus-host, healing after surgical procedures, post radiation atrophy and chemotherapy, and osteoporosis.

It is therefore an object of the present invention to provide methods of treating the above diseases, the method comprising administering to a subject in need thereof a therapeutically effective amount of a GLP-2 or GLP-2 compound conjugate according to this invention. The term “GLP-2 compound” comprises analogues and derivatives of GLP-2 with GLP-2 activity

Growth hormone may be used in the treatment of growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1st toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Crohn's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteoarthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; or short stature due to glucocorticoid treatment in children.

Growth hormones have also been used for acceleration of the healing of muscle tissue, nervous tissue or wounds; the acceleration or improvement of blood flow to damaged tissue; or the decrease of infection rate in damaged tissue; or the decrease of infection rate in damaged tissue, the method comprising administration to a patient in need thereof an effective amount of a therapeutically effective amount of a therapeutic peptide as defined herein.

The present invention thus provides a method for treating these disorders, diseases or states, the method comprising administering to a patient in need thereof a therapeutically effective amount of a growth hormone or growth hormone compound conjugate according to the present invention. The term “growth hormone compound” comprises analogues and derivatives of growth hormone with growth hormone activity.

Cytokines are implicated in the aetiology of a host of diseases involving the immune system. In particular it is mentioned that IL-20 could be involved in psoriasis and its treatment, and IL-21 is involved in cancer and could constitute a treatment to this disease.

The present invention thus provides a method for treating these disorders, diseases or states, comprising the administration of a therapeutically effective amount of an IL-20 or IL-20 compound conjugate according to the present invention. The term “IL-20 compound” comprises analogues and derivatives of IL-20 with IL-20 activity.

TTF peptides may be used to increase the viscosity of mucus layers in subject, to reduce secretion of saliva, e.g. where the increased saliva secretion is caused by irradiation therapy, treatment with anticholinergics or Sjögren's syndrome, to treat allergic rhinitis, stress induced gastric ulcers secondary to trauma, shock, large operations, renal or liver diseases, treatment with NSAID, e.g. aspirin, steroids or alcohol. TTF peptides may also be used to treat Chrohn's disease, ulcerative colitis, keratoconjunctivitis, chronic bladder infections, intestinal cystitis, papillomas and bladder cancer.

In one embodiment, the invention thus relates to a method of treating the above mention diseases or states, the method comprising administering to a subject patient in need thereof a therapeutically effective amount of a TTF conjugate or TTF compound conjugate according to the present invention. The term “TTF compound” comprises analogues and derivatives of TTF with TTF activity.

Melanocortin receptor modifiers, and in particular melanocortin 4 receptor agonists have been implicated in the treatment and prevention of obesity and related diseases. Melanocortin 4 receptor agonists have also been implicated in the treatment of diseases selected from atherosclerosis, hypertension, diabetes, type 2 diabetes, impaired glucose tolerance (IGT), dyslipidemia, coronary heart disease, gallbladder disease, gall stone, osteoarthritis, cancer, sexual dysfunction and the risk of premature death.

In one embodiment, the invention thus provides a method of treating the above diseases or states, the method comprising administering to a subject in need thereof a therapeutically effective amount of an melanocortin 4 receptor agonist conjugate or melanocortin 4 receptor agonist compound conjugate of the present invention. The term “melanocortin 4 receptor” comprises analogues and derivatives of melanocortin 4 receptor with melanocortin 4 receptor activity.

Factor VII compounds have been implicated in the treatment of disease related to coagulation, and biological active Factor VII compounds in particular have been implicated in the treatment of haemophiliacs, haemophiliacs with inhibitors to Factor VIII and IX, patients with thrombocytopenia, patients with thrombocytopathies, such as Glanzmann's thrombastenia platelet release defect and storage pool defects, patient with von Willebrand's disease, patients with liver disease and bleeding problems associated with traumas or surgery. Biologically inactive Factor VII compounds have been implicated in the treatment of patients being in hypercoagluable states, such as patients with sepsis, deep vein thrombosis, patients in risk of myocardial infections or thrombotic stroke, pulmonary embolism, patients with acute coronary syndromes, patients undergoing coronary cardiac, prevention of cardiac events and restenosis for patient receiving angioplasty, patient with peripheral vascular diseases, and acute respiratory distress syndrome.

In one embodiment, the invention thus provides a method for the treatment of the above-mentioned diseases or states, the method comprising administering to a subject in need thereof a therapeutically effective amount of a Factor VII conjugate or Factor VII compound conjugate according to the present invention. The term “Factor VII compound” comprises analogues and derivatives of Factor VII with Factor VII activity.

In one embodiment, the invention provides the use of the therapeutic peptides, as hereinbefore described, conjugated according to the methods of the present invention in the manufacture of a medicament used in the treatment of the above-mentioned diseases or states.

In one embodiment, there is provided a pharmaceutical composition comprising the therapeutic peptides, as hereinbefore described, conjugated according to the methods of the present invention for use in the treatment of the diseases, disorders or conditions as hereinbefore described.

Many diseases are treated using more than one medicament in the treatment, either concomitantly administered or sequentially administered. It is therefore within the scope of the present invention to use the peptide conjugates of the present invention in therapeutic methods for the treatment of one of the above mentioned diseases in combination with one another, or as an adjunct to, or in conjunction with, other established therapies normally used in the treatment of said disease. By analogy, it is also within the scope of the present invention to use the peptide conjugates of the present invention in combination with other therapeutically active compounds normally used in the treatment of one of the above mentioned diseases in the manufacture of a medicament for said disease.

Another purpose is to provide a pharmaceutical composition comprising a conjugated peptide, such as conjugated growth hormone (GH) of the present invention which is present in a concentration from 10-15 mg/ml to 200 mg/ml, such as e.g. 10-10 mg/ml to 5 mg/ml and wherein said composition has a pH from 2.0 to 10.0. The composition may further comprise a buffer system, preservative(s), tonicity agent(s), chelating agent(s), stabilizers and surfactants.

In one embodiment of the invention the pharmaceutical composition is an aqueous composition, i.e. a composition comprising water. Such a composition is typically a solution or a suspension. In a further embodiment of the invention the pharmaceutical composition is an aqueous solution. The term “aqueous composition” is defined as a composition comprising at least 50% w/w water. Likewise, the term “aqueous solution” is defined as a solution comprising at least 50% w/w water, and the term “aqueous suspension” is defined as a suspension comprising at least 50% w/w water.

In one embodiment the pharmaceutical composition is a freeze-dried composition, whereto the physician or the patient adds solvents and/or diluents prior to use.

In one embodiment the pharmaceutical composition is a dried composition (e.g. freeze-dried or spray-dried) ready for use without any prior dissolution.

In one embodiment the invention relates to a pharmaceutical composition comprising an aqueous solution of a peptide conjugate, such as e.g. a GH conjugate, and a buffer, wherein said peptide conjugate, such as e.g. GH conjugate is present in a concentration from 0.1-100 mg/ml or above, and wherein said composition has a pH from about 2.0 to about 10.0.

In one embodiment of the invention the pH of the composition is selected from the list consisting of 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, and 10.0.

In one embodiment of the invention the buffer is selected from the group consisting of sodium acetate, sodium carbonate, citrate, glycylglycine, histidine, glycine, lysine, arginine, sodium dihydrogen phosphate, disodium hydrogen phosphate, sodium phosphate, and tris(hydroxymethyl)-aminomethan, bicine, tricine, malic acid, succinate, maleic acid, fumaric acid, tartaric acid, aspartic acid or mixtures thereof. Each one of these specific buffers constitutes an alternative embodiment of the invention.

In one embodiment of the invention the composition further comprises a pharmaceutically acceptable preservative. The use of a preservative in pharmaceutical compositions is well known to the skilled person. For convenience reference is made to Remington: The Science and Practice of Pharmacy, 20th edition, 2000.

In one embodiment of the invention the composition further comprises an isotonic agent. The use of an isotonic agent in pharmaceutical compositions is well known to the skilled person. For convenience reference is made to Remington: The Science and Practice of Pharmacy, 20th edition, 2000.

In one embodiment of the invention the composition further comprises a chelating agent. The use of a chelating agent in pharmaceutical compositions is well known to the skilled person. For convenience reference is made to Remington: The Science and Practice of Pharmacy, 20th edition, 2000.

In one embodiment of the invention the composition further comprises a stabiliser. The use of a stabilizer in pharmaceutical compositions is well known to the skilled person. For convenience reference is made to Remington: The Science and Practice of Pharmacy, 20th edition, 2000.

More particularly, compositions of the invention are stabilized liquid pharmaceutical compositions whose therapeutically active components include a protein that possibly exhibits aggregate formation during storage in liquid pharmaceutical compositions. By “aggregate formation” is intended a physical interaction between the protein molecules that results in formation of oligomers, which may remain soluble, or large visible aggregates that precipitate from the solution. By “during storage” is intended a liquid pharmaceutical composition or composition once prepared, is not immediately administered to a subject. Rather, following preparation, it is packaged for storage, either in a liquid form, in a frozen state, or in a dried form for later reconstitution into a liquid form or other form suitable for administration to a subject.

The term “stabilized composition” refers to a composition with increased physical stability, increased chemical stability or increased physical and chemical stability. In general, a composition must be stable during use and storage (in compliance with recommended use and storage conditions) until the expiration date is reached.

The term “physical stability” of the protein composition as used herein refers to the tendency of the protein to form biologically inactive and/or insoluble aggregates of the protein as a result of exposure of the protein to thermo-mechanical stresses and/or interaction with interfaces and surfaces that are destabilizing, such as hydrophobic surfaces and interfaces.

The term “chemical stability” of the protein composition as used herein refers to chemical covalent changes in the protein structure leading to formation of chemical degradation products with potential less biological potency and/or potential increased immunogenic properties compared to the native protein structure. Various chemical degradation products can be formed depending on the type and nature of the native protein and the environment to which the protein is exposed.

By “dried form” is intended the liquid pharmaceutical composition or composition is dried either by freeze drying (i.e., lyophilization; see, for example, Williams and Polli (1984) J. Parenteral Sci. Technol. 38:48-59), spray drying (see Masters (1991) in Spray-Drying Handbook (5th ed; Longman Scientific and Technical, Essez, U.K.), pp. 491-676; Broadhead et al. (1992) Drug Devel. Ind. Pharm. 18:1169-1206; and Mumenthaler et al. (1994) Pharm. Res. 11:12-20), or air drying (Carpenter and Crowe (1988) Cryobiology 25:459-470; and Roser (1991) Biopharm. 4:47-53).

Aggregate formation by a protein during storage of a liquid pharmaceutical composition can adversely affect biological activity of that protein, resulting in loss of therapeutic efficacy of the pharmaceutical composition. Furthermore, aggregate formation may cause other problems such as blockage of tubing, membranes, or pumps when the protein-containing pharmaceutical composition is administered using an infusion system.

The pharmaceutical compositions of the invention may further comprise an amount of an amino acid base sufficient to decrease aggregate formation by the protein during storage of the composition. By “amino acid base” is intended an amino acid or a combination of amino acids, where any given amino acid is present either in its free base form or in its salt form. Where a combination of amino acids is used, all of the amino acids may be present in their free base forms, all may be present in their salt forms, or some may be present in their free base forms while others are present in their salt forms. In one embodiment, amino acids to use in preparing the compositions of the invention are those carrying a charged side chain, such as arginine, lysine, aspartic acid, and glutamic acid. Any stereoisomer (i.e., L or D isomer, or mixtures thereof) of a particular amino acid (methionine, histidine, arginine, lysine, isoleucine, aspartic acid, tryptophan, threonine and mixtures thereof) or combinations of these stereoisomers or glycine or an organic base such as but not limited to imidazole, may be present in the pharmaceutical compositions of the invention so long as the particular amino acid or organic base is present either in its free base form or its salt form. In one embodiment the L-stereoisomer of an amino acid is used.

Compositions of the invention may also be formulated with analogues of these amino acids. By “amino acid analogue” is intended a derivative of the naturally occurring amino acid that brings about the desired effect of decreasing aggregate formation by the protein during storage of the liquid pharmaceutical compositions of the invention. Suitable arginine analogues include, for example, aminoguanidine, ornithine and N-monoethyl L-arginine, suitable methionine analogues include ethionine and buthionine and suitable cysteine analogues include S-methyl-L cysteine. As with other amino acids, the amino acid analogues are incorporated into the compositions in either their free base form or their salt form. In a further embodiment of the invention the amino acids or amino acid analogues are used in a concentration, which is sufficient to prevent or delay aggregation of the protein.

In one embodiment of the invention methionine (or other sulphuric amino acids or amino acid analogous) may be added to inhibit oxidation of methionine residues to methionine sulfoxide when the protein acting as the therapeutic agent is a protein comprising at least one methionine residue susceptible to such oxidation. By “inhibit” is intended minimal accumulation of methionine oxidized species over time. Inhibiting methionine oxidation results in greater retention of the protein in its proper molecular form. Any stereoisomer of methionine (L or D isomer) or any combinations thereof can be used. The amount to be added should be an amount sufficient to inhibit oxidation of the methionine residues such that the amount of methionine sulfoxide is acceptable to regulatory agencies. Typically, this means that the composition contains no more than about 10% to about 30% methionine sulfoxide. Generally, this can be obtained by adding methionine such that the ratio of methionine added to methionine residues ranges from about 1:1 to about 1000:1, such as 10:1 to about 100:1.

In one embodiment of the invention the composition further comprises a stabilizer selected from the group of high molecular weight polymers or low molecular compounds. In a further embodiment of the invention the stabilizer is selected from polyethylene glycol (e.g. PEG 3350), polyvinyl alcohol (PVA), polyvinylpyrrolidone, carboxy/hydroxycellulose or derivates thereof (e.g. HPC, HPC-SL, HPC-L and HPMC), cyclodextrins, sulphur-containing substances as monothioglycerol, thioglycolic acid and 2-methylthioethanol, and different salts (e.g. sodium chloride). Each one of these specific stabilizers constitutes an alternative embodiment of the invention.

The pharmaceutical compositions may also comprise additional stabilizing agents, which further enhance stability of a therapeutically active protein therein. Stabilizing agents of particular interest to the present invention include, but are not limited to, methionine and EDTA, which protect the protein against methionine oxidation, and a non-ionic surfactant, which protects the protein against aggregation associated with freeze-thawing or mechanical shearing.

In one embodiment of the invention the composition further comprises a surfactant. The use of a surfactant in pharmaceutical compositions is well-known to the skilled person. For convenience reference is made to Remington: The Science and Practice of Pharmacy, 20th edition, 2000.

It is possible that other ingredients may be present in the pharmaceutical composition of the present invention. Such additional ingredients may include wetting agents, emulsifiers, antioxidants, bulking agents, tonicity modifiers, chelating agents, metal ions, oleaginous vehicles, proteins (e.g., human serum albumin, gelatine or proteins) and a zwitterion (e.g., an amino acid such as betaine, taurine, arginine, glycine, lysine and histidine). Such additional ingredients, of course, should not adversely affect the overall stability of the pharmaceutical composition of the present invention.

Pharmaceutical compositions containing a peptide conjugate, such as e.g. a GH conjugate according to the present invention may be administered to a patient in need of such treatment at several sites, for example, at topical sites, for example, skin and mucosal sites, at sites which bypass absorption, for example, administration in an artery, in a vein, in the heart, and at sites which involve absorption, for example, administration in the skin, under the skin, in a muscle or in the abdomen.

Administration of pharmaceutical compositions according to the invention may be through several routes of administration, for example, oral, rectal, nasal, pulmonary, topical (including buccal and sublingual), transdermal, intracisternal, intraperitoneal, vaginal and parenteral (including subcutaneous, intramuscular, intrathecal, intravenous and intradermal) route. It will be appreciated that the preferred route will depend on the general condition and age of the subject to be treated, the nature of the condition to be treated and the active ingredient chosen.

Compositions of the current invention may be administered in several dosage forms, for example, as solutions, suspensions, emulsions, microemulsions, multiple emulsion, foams, salves, pastes, plasters, ointments, tablets, coated tablets, rinses, capsules, for example, hard gelatine capsules and soft gelatine capsules, suppositories, rectal capsules, drops, gels, sprays, powder, aerosols, inhalants, eye drops, ophthalmic ointments, ophthalmic rinses, vaginal pessaries, vaginal rings, vaginal ointments, injection solution, in situ transforming solutions, for example in situ gelling, in situ setting, in situ precipitating, in situ crystallization, infusion solution, and implants.

Compositions of the invention may further be compounded in, or attached to, for example through covalent, hydrophobic and electrostatic interactions, a drug carrier, drug delivery system and advanced drug delivery system in order to further enhance stability of the GH conjugate, increase bioavailability, increase solubility, decrease adverse effects, achieve chronotherapy well known to those skilled in the art, and increase patient compliance or any combination thereof. Examples of carriers, drug delivery systems and advanced drug delivery systems include, but are not limited to, polymers, for example cellulose and derivatives, polysaccharides, for example dextran and derivatives, starch and derivatives, poly(vinyl alcohol), acrylate and methacrylate polymers, polylactic and polyglycolic acid and block co-polymers thereof, polyethylene glycols, carrier proteins, for example albumin, gels, for example, thermogelling systems, for example block co-polymeric systems well known to those skilled in the art, micelles, liposomes, microspheres, nanoparticulates, liquid crystals and dispersions thereof, L2 phase and dispersions there of, well known to those skilled in the art of phase behaviour in lipid-water systems, polymeric micelles, multiple emulsions, self-emulsifying, self-microemulsifying, cyclodextrins and derivatives thereof, and dendrimers.

Compositions of the current invention are useful in the composition of solids, semi-solids, powder and solutions for pulmonary administration of a peptide conjugate, such as e.g. a GH conjugate, using, for example a metered dose inhaler, dry powder inhaler and a nebulizer, all being devices well known to those skilled in the art.

Compositions of the current invention are specifically useful in the composition of controlled, sustained, protracting, retarded, and slow release drug delivery systems. More specifically, but not limited to, compositions are useful in composition of parenteral controlled release and sustained release systems (both systems leading to a many-fold reduction in number of administrations), well known to those skilled in the art. Even more preferably, are controlled release and sustained release systems administered subcutaneous. Without limiting the scope of the invention, examples of useful controlled release system and compositions are hydrogels, oleaginous gels, liquid crystals, polymeric micelles, microspheres, nanoparticles.

Methods to produce controlled release systems useful for compositions of the current invention include, but are not limited to, crystallization, condensation, co-crystallization, precipitation, co-precipitation, emulsification, dispersion, high pressure homogenisation, encapsulation, spray drying, microencapsulating, coacervation, phase separation, solvent evaporation to produce microspheres, extrusion and supercritical fluid processes. General reference is made to Handbook of Pharmaceutical Controlled Release (Wise, D. L., ed. Marcel Dekker, New York, 2000) and Drug and the Pharmaceutical Sciences vol. 99: Protein Composition and Delivery (MacNally, E. J., ed. Marcel Dekker, New York, 2000).

Parenteral administration may be performed by subcutaneous, intramuscular, intraperitoneal or intravenous injection by means of a syringe, optionally a pen-like syringe. Alternatively, parenteral administration can be performed by means of an infusion pump. A further option is a composition which may be a solution or suspension for the administration of the peptide conjugate, such as e.g. the GH conjugate in the form of a nasal or pulmonal spray. As a still further option, the pharmaceutical compositions containing the peptide conjugate, such as e.g. the GH conjugate of the invention can also be adapted to transdermal administration, e.g. by needle-free injection or from a patch, optionally an iontophoretic patch, or transmucosal, e.g. buccal, administration.

In one embodiment of the invention the pharmaceutical composition comprising the conjugate is stable for more than 6 weeks of usage and for more than three years of storage.

In one embodiment of the invention the pharmaceutical composition comprising the conjugate is stable for more than 4 weeks of usage and for more than three years of storage.

In one embodiment of the invention the pharmaceutical composition comprising the conjugate is stable for more than 4 weeks of usage and for more than two years of storage.

In one embodiment of the invention the pharmaceutical composition comprising the conjugate is stable for more than 2 weeks of usage and for more than two years of storage.

The conjugate peptides, or compositions thereof, will generally be used in an amount effective to achieve the intended result, for example in an amount effective to treat or prevent the particular disease being treated. The compound(s) may be administered therapeutically to achieve therapeutic benefit. By therapeutic benefit is meant eradication or amelioration of the underlying disorder being treated and/or eradication or amelioration of one or more of the systems associated with the underlying disorder. Therapeutic benefit also includes halting or slowing the progression of the disease, regardless of whether improvement is realised.

The exact dosage will depend upon the frequency and mode of administration, the sex, age, weight and general condition of the subject treated, the nature and severity of the condition treated and any concomitant diseases to be treated and other factors evident to those skilled in the art. It will be understood that determining an appropriate dosage may be achieved using routine experimentation, by constructing a matrix of values and testing different points in the matrix, which is all within the ordinary skills of a trained physician or veterinary.

When a conjugate peptide or a pharmaceutically acceptable salt, solvate or prodrug thereof is used in combination with a second therapeutic agent active against the same disease state the dose of each compound may differ from that when the compound is used alone. Appropriate doses will be readily appreciated by those skilled in the art.

All references, including publications, patent applications, and patents, cited herein are hereby incorporated by reference in their entirety and to the same extent as if each reference were individually and specifically indicated to be incorporated by reference and were set forth in its entirety herein (to the maximum extent permitted by law).

All headings and sub-headings are used herein for convenience only and should not be construed as limiting the invention in any way.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### EXAMPLES

The invention will be further defined by reference to the following examples. It will be apparent to those skilled in the art that many modifications, both to the materials and methods may be practiced without departing from the scope of the invention.

### Abbreviations

TGase:
(a) TGase Activa WM from Ajinomoto, 60 mg/ml in 20 mM Phosphate buffer pH 8.5. The Activa WM enzyme from Streptoverticillium mobaraense is formulated under dry form in maltodextrins and contains 1% w/w of protein) (6.7 μl)); or
(b) TGase MTG P2 from Streptovercilium mobaraense expressed in soluble form using E. coli as expression system was formulated in 20 mM NaPhosphate buffer, pH 6.0, with 2 mM DTT and 5 w/v % maltodextrin. The concentration of the enzyme was between 0.7 and 4.5 mg/ml.

### Analytical Methods

### Capillary Electrophoresis

Capillary electrophoresis (CE) was carried out using an Agilent Technologies 3D-CE system (Agilent Technologies). Data acquisition and signal processing were performed using Agilent Technologies 3DCE ChemStation. The capillary was a 64.5 cm (56.0 cm efficient length) 50 μm i.d. “Extended Light Path Capillary” from Agilent. UV detection was performed at 200 nm (16 nm Bw, Reference 380 nm and 50 nm Bw). The running electrolyte was phosphate buffer 50 mM pH7.0. The capillary was conditioned with 0.1M NaOH for 3 min, then with Milli-Q water for 2 min and with the electrolyte for 3 min.

After each run, the capillary was flushed with milli-Q water for 2 min, then with phosphoric acid for 2 min, and with milli-Q water for 2 min. The hydrodynamic injection was done at 50 mbar for 4.0 s. The voltage was +25 kV. The capillary temperature was 30 C and the runtime was 10.5 min.

### Illustrative Scheme for the Conjugation of hGH

N^{ε141}-[2-(4-(4-(40 KDa mPEGyl)butanoyl)-amino-butyloxyimino)-ethyl] hGH is synthesized according to the following scheme:

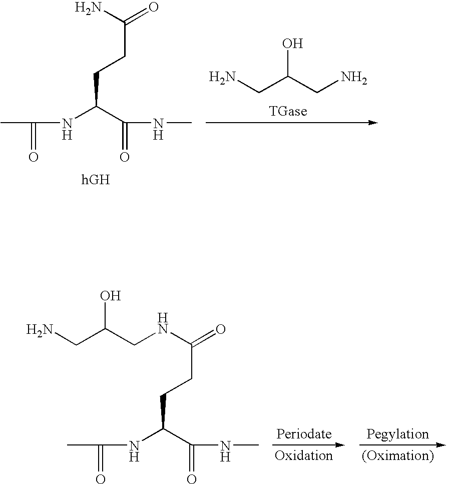

### Method of TGase Catalyzed Step

To a 1M solution of 1,3-diamino-1-propanol in triethanolamine 20 mM pH8.6 buffer (14 μl) was added triethanolamine 20 mM pH8.6 buffer (20.6 μl), ethylene glycol (53.6 μl) (40% w/v)), and a solution of human growth hormone (50 mg/ml) in triethanolamine 20 mM pH8.6 buffer (40 μl). The reaction was started by the addition of the TGase MTG P2 solution (0.35 mg/ml, 5.8 μl). The reaction mixture was incubated at ambient temperature and followed on CE.

### Example 1

### Transamination of hGH in the Presence and in the Absence of Ethylene Glycol

The reaction was carried out in an analogous manner to the above method using the following concentrations:
[hGH]=0.67 mM (14.9 mg/ml final concentration)
[1,3-diamino 2-propanol]=300 mM
Buffer: 20 mM Triethanolamine pH 8.6
+/−Ethylene glycol 40% (v/v)
[TGase MTG P2]=0.39 μM

Total volume: 134 μl

Ambient temperature

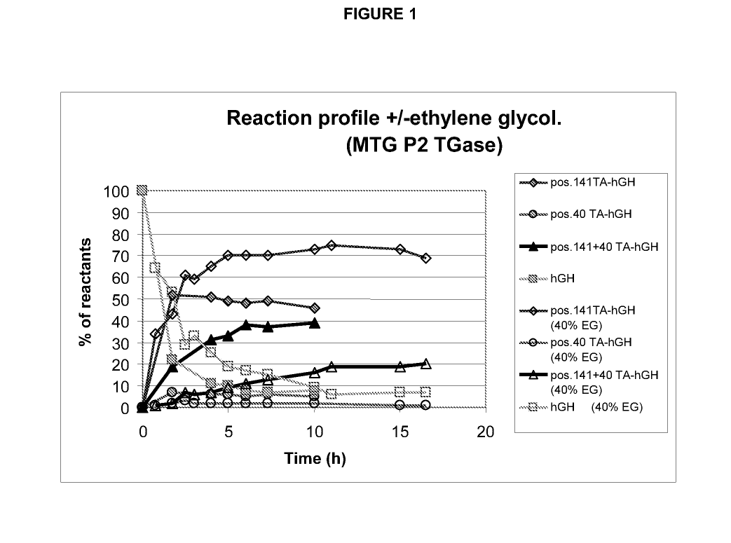

The results shown in FIG. 1 demonstrate that a maximum yield of 75% of the desired pos.141-hGH was obtained after 11 h. In the control experiment, the maximum yield of 53% of the desired product was obtained after 4 h reaction time.

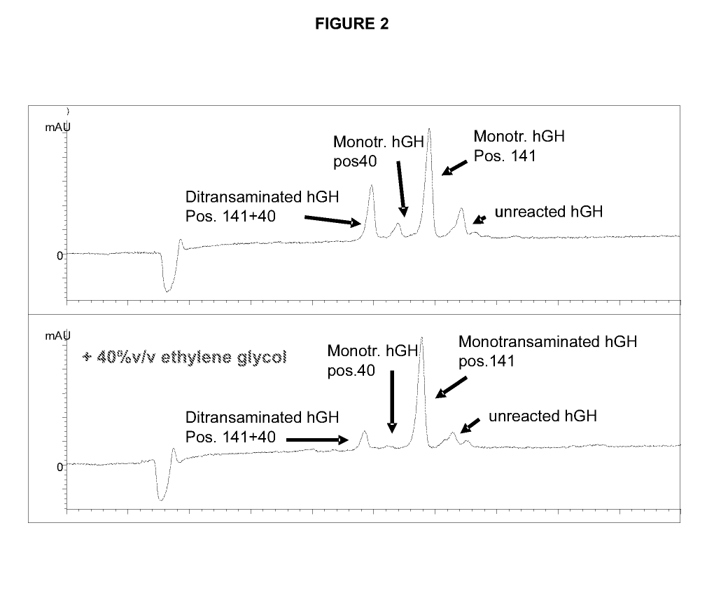

Electropherograms were obtained from the reaction mixtures taken at reaction times when the desired product was at its maximum yield, i.e. 4 h for the reaction mixture without ethylene glycol and 11 h for the reaction mixture containing 40% v/v ethylene glycol. The results are shown in FIG. 2.

### Example 2

### Comparing the Effect of Ethylene Glycol Using Either TGase Activa WM or TGase MTG P2

The reaction was carried out in an analogous manner to the above method using the following concentrations:
[hGH]=0.67 mM (14.9 mg/ml final concentration)
[1,3-diamino 2-propanol]=300 mM
Buffer: 20 mM Triethanolamine pH 8.6
+/−Ethylene glycol 40% (v/v)
[TGase MTG P2]=0.39 μM or [TGase Activa WM]=1.95 μM

Ambient temperature

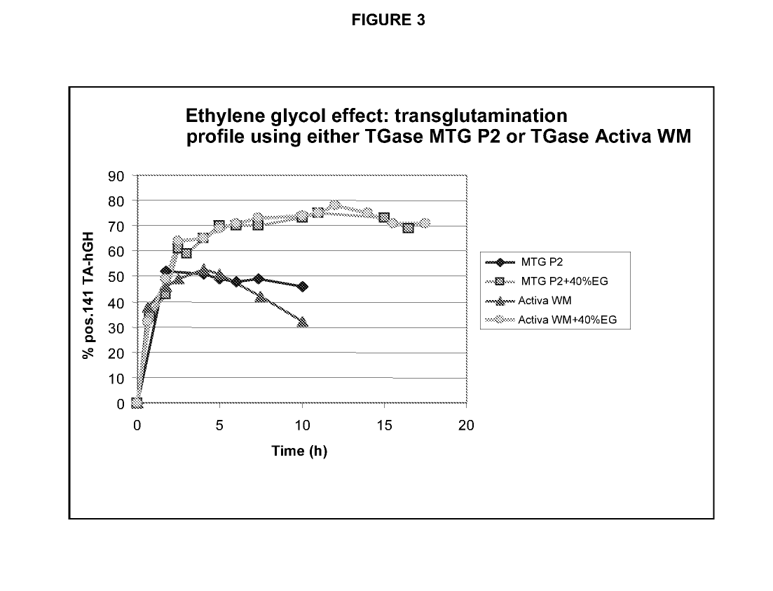

The results shown in FIG. 3 demonstrate that the reaction profiles of both enzymes are very similar.

### Example 3

### Transamination of hGH in the Presence of 10 to 40% v/v Ethylene Glycol

The reaction was carried out in an analogous manner to the above method using the following concentrations:
[hGH]=0.67 mM (14.9 mg/ml final concentration)
[1,3-diamino 2-propanol]=300 mM
Buffer: 20 mM Triethanolamine pH 8.6
+/−Ethylene glycol 10-40% (v/v)
[TGase MTG P2]=0.39 μM

Total volume: 134 μl

Incubation at ambient temperature

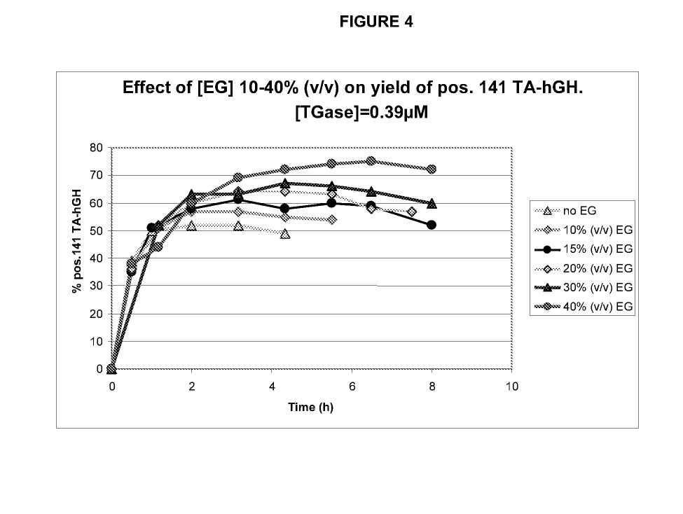

The results illustrated in FIG. 4 reveal that an increased yield of pos.141 TA-hGH was obtained when the concentration of ethylene glycol was increased.

### Example 4

### Transamination of hGH in the Presence of 40 to 70% v/v Ethylene Glycol

The reaction was carried out in an analogous manner to the above method, except for the fact that more enzyme was added after five hours of reaction time. The reaction used the following concentrations:
[hGH]=0.67 mM (14.9 mg/ml final concentration)
[1,3-diamino 2-propanol]=300 mM
Buffer: 20 mM Triethanolamine pH 8.6
+/−Ethylene glycol 40-70% (v/v)
[TGase MTG P2]=0.39 μM then 0.78 μM at 5 hours

Total volume: 134 μl

Ambient temperature

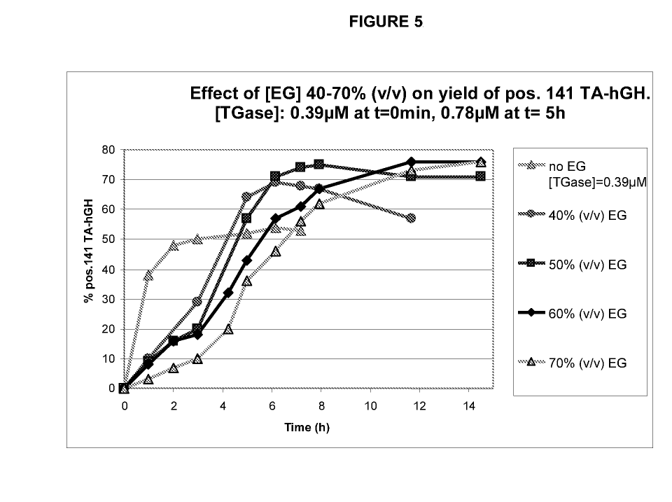

The results are shown in FIG. 5.

### Example 5

### Comparing the Effect of Ethylene Glycol Using Varying Concentrations of TGase MTG P2

The reaction was carried out in an analogous manner to the above method using the following concentrations:
[hGH]=0.67 mM (14.9 mg/ml final concentration)
[1,3-diamino 2-propanol]=300 mM
Buffer: 20 mM Triethanolamine pH 8.6
+/−Ethylene glycol 40% (v/v)
[TGase MTG P2]=0.39 μM or 0.16 μM

Ambient temperature

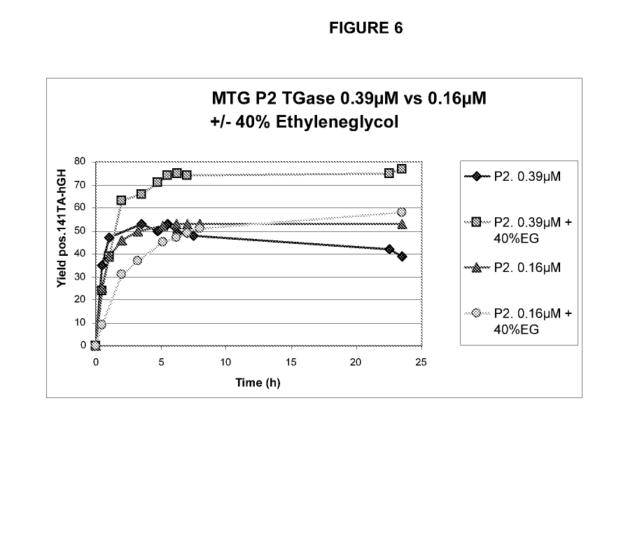

The results shown in FIG. 6 demonstrate that 0.39 μM MTG P2 enzyme and 40% ethylene glycol gave the highest product yield.

### Example 6

### Comparing the Effect of Compounds of Related Structures

The reaction was carried out with ethylene glycol, glycerol, propylene glycol and 1,3 propanediol in an analogous manner to the above method using the following concentrations:
[hGH]=0.67 mM (14.9 mg/ml final concentration)
[1,3-diamino 2-propanol]=300 mM
Buffer: 20 mM Triethanolamine pH 8.6
+/−enhancing compound 7.18 M
[TGase MTG P2]=0.39 μM

Total volume: 134 μl

Ambient temperature

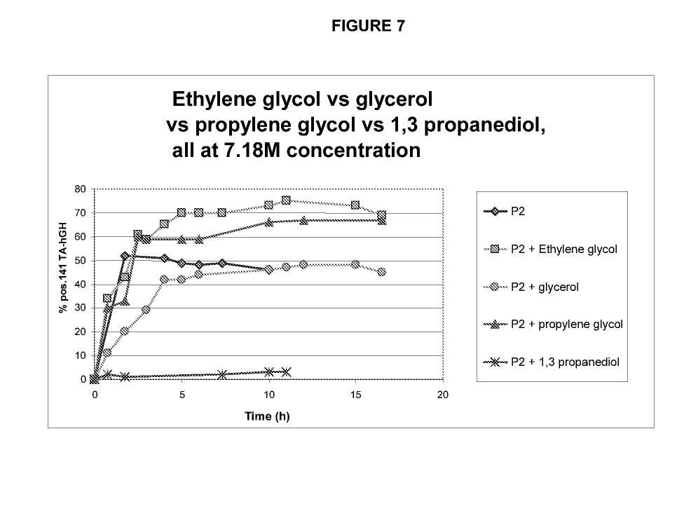

The results shown in FIG. 7 demonstrate that ethylene glycol gave the highest yield. Propylene glycol provided the next highest yield. Glycerol was found to reduce the amount of side products (pos.40-TA-hGH and di-transaminated hGH) formed, thus glycerol also improved the selectivity of the reaction.

1,3 propanediol completely inhibited the reaction.

### Example 7

### Comparing the Effect of Glycerol Using Varying Concentrations of TGase MTG P2

The reaction was carried out with glycerol in an analogous manner to the above method, except for the fact that more enzyme was added after five hours of reaction time. The reaction used the following concentrations:
[hGH]=0.67 mM (14.9 mg/ml final concentration)
[1,3-diamino 2-propanol]=300 mM
Buffer: 20 mM Triethanolamine pH 8.6
Glycerol 7.18 M
[TGase MTG P2]=0.39 μM then 1.17 μM at 5 hours

Total volume: 134 μl

Ambient temperature

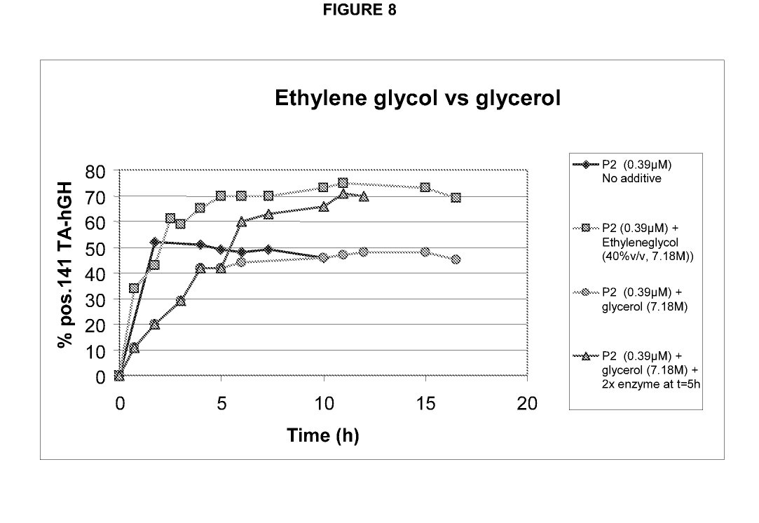

The results shown in FIG. 8 demonstrate that the yield obtained with glycerol can be increased when more enzyme is added to the reaction mixture.

1. A method for enhancing the selectivity and/or yield of a transglutaminase-catalysed reaction, which transglutaminase-catalysed reaction comprises covalent bond formation between a first compound and a peptide, and said method comprises reacting in one or more steps the first compound with the peptide in the presence of said transglutaminase and an enhancing compound, wherein said enhancing compound is selected from one of ethylene glycol, propylene glycol or glycerol.

2. The method according to claim 1, wherein the peptide is selected from insulin, glucagon like-peptide 1 (GLP-1), glucagon like-peptide 2 (GLP-20, prolactin, growth hormone, an interleukin, a cytokine, an antibody, TFF, a melanocortin receptor modifier, a coagulation factor and a factor VII compound.

3. The method according to claim 2, wherein the peptide is growth hormone, e.g. human growth hormone.

4. A method for preparing a growth hormone conjugate, wherein said method comprises reacting in one or more steps a first compound with the growth hormone in the presence of said a transglutaminase, wherein said reaction is performed in the presence of an enhancing compound, wherein said enhancing compound is selected from one of ethylene glycol, propylene glycol or glycerol.

5. The method according to claim 3 wherein the yield of growth hormone having the first compound covalently attached to the glutamine residue in the position corresponding to position 141 of human growth hormone is increased.

6. The method according to claim 1, wherein the enhancing compound is ethylene glycol.

7. The method according to claim 1, wherein the first compound is represented by the formula:
H₂N-D-R—X
wherein D represents a bond or oxygen;
R represents a linker or a bond;
X is selected from or can be activated to keto-, aldehyde-, —NH—NH₂, —O—C(O)—NH—NH₂, —NH—C(O)—NH—NH₂, —NH—C(S)—NH—NH₂, —NHC(O)—NH—NH—C(O)—NH—NH₂, —NH—NH—C(O)—NH—NH₂, —NH—NH—C(S)—NH—NH₂, —NH—C(O)—C₆H₄—NH—NH₂, —C(O)—NH—NH₂, —O—NH₂, —C(O)—O—NH₂, —NH—C(O)—O—NH₂, —NH—C(S)—O—NH₂, C(O)—NH₂, Ar—NH₂, alkyne, azide or nitril-oxide.

8. The method according to claim 1, wherein the first compound comprises one or more functional groups or latent functional groups which are not accessible in any of the amino acid residues constituting said peptide, and the method additionally comprises the steps of:
(b) optionally activating the latent functional group; and
(c) reacting in one or more steps said functionalised peptide with a second compound comprising one or more functional groups, wherein said functional group(s) do not react with functional groups accessible in the amino acid residues constituting said peptide, and wherein said functional group(s) in said second compound is capable of reacting with said functional group(s) in said first compound so that a covalent bond between said functionalised peptide and second compound is formed.

9. The method according to claim 1 wherein said first compound is selected from 4-(aminomethyl)phenyl ethanone, 4-(2-aminoethyl)phenyl ethanone, N-(4-acetylphenyl) 2-aminoacetamide, 1-[4-(2-aminoethoxy)phenyl]ethanone, 1-[3-(2-aminoethoxy)phenyl]ethanone, 1,4-bis(aminoxy)butane, 3-oxapentane-1,5-dioxyamine, 1,8-diaminoxy-3,6-dioxaoctane, 1,3-bis(aminoxy)propan-2-ol, 1,11-bis(aminoxy)-3,6,9-trioxaundecane, 1,3-diamino-2-propanol, 1,2-bis(aminoxy)ethane, and 1,3-bis(aminoxy)propane.

10. A method for enhancing the selectivity and/or yield of a transglutaminase-catalysed reaction, which transglutaminase-catalysed reaction comprises covalent bond formation between a first compound and a growth hormone, and wherein the yield, which is increased is the yield of growth hormone conjugated in the position corresponding to position 141 of human growth hormone, said method comprising reacting in one or more steps the first compound with the growth hormone in the presence of said transglutaminase, wherein said method is characterizing in that the reaction of the first compound with the growth hormone in the presence of said transglutaminase takes place in the presence of an enhancing compound, wherein said enhancing compound is selected from one of ethylene glycol, propylene glycol or glycerol.

11. The method according to claim 1, wherein the reaction is buffered to a pH between 6 and 9.

12. The method according to claim 11, wherein the reaction is buffered to a pH between 7 and 8.6.

13. The method according to claim 1, wherein the reaction is carried out at a temperature between 15 and 45° C.

14. The method according to claim 13 wherein the reaction is carried out at a temperature between 20 and 37° C.

15. The method according to claim 1, wherein the enhancing compound is present in a concentration range between 20 to 95%.

16. The method according to claim 15, wherein the enhancing compound is present in a concentration range between 30 to 70%.

17. A peptide obtainable by a method according to claim 1.

18. A method treatment of growth hormone deficiency (GHD); Turner Syndrome; Prader-Willi syndrome (PWS); Noonan syndrome; Down syndrome; chronic renal disease, juvenile rheumatoid arthritis; cystic fibrosis, HIV-infection in children receiving HAART treatment (HIV/HALS children); short children born short for gestational age (SGA); short stature in children born with very low birth weight (VLBW) but SGA; skeletal dysplasia; hypochondroplasia; achondroplasia; idiopathic short stature (ISS); GHD in adults; fractures in or of long bones, such as tibia, fibula, femur, humerus, radius, ulna, clavicula, matacarpea, matatarsea, and digit; fractures in or of spongious bones, such as the scull, base of hand, and base of food; patients after tendon or ligament surgery in e.g. hand, knee, or shoulder; patients having or going through distraction oteogenesis; patients after hip or discus replacement, meniscus repair, spinal fusions or prosthesis fixation, such as in the knee, hip, shoulder, elbow, wrist or jaw; patients into which osteosynthesis material, such as nails, screws and plates, have been fixed; patients with non-union or mal-union of fractures; patients after osteatomia, e.g. from tibia or 1st toe; patients after graft implantation; articular cartilage degeneration in knee caused by trauma or arthritis; osteoporosis in patients with Turner syndrome; osteoporosis in men; adult patients in chronic dialysis (APCD); malnutritional associated cardiovascular disease in APCD; reversal of cachexia in APCD; cancer in APCD; chronic abstractive pulmonal disease in APCD; HIV in APCD; elderly with APCD; chronic liver disease in APCD, fatigue syndrome in APCD; Crohn's disease; impaired liver function; males with HIV infections; short bowel syndrome; central obesity; HIV-associated lipodystrophy syndrome (HALS); male infertility; patients after major elective surgery, alcohol/drug detoxification or neurological trauma; aging; frail elderly; osteo-arthritis; traumatically damaged cartilage; erectile dysfunction; fibromyalgia; memory disorders; depression; traumatic brain injury; subarachnoid haemorrhage; very low birth weight; metabolic syndrome; glucocorticoid myopathy; or short stature due to glucocorticoid treatment in children. Growth hormones have also been used for acceleration of the healing of muscle tissue, nervous tissue or wounds; the acceleration or improvement of blood flow to damaged tissue; or the decrease of infection rate in damaged tissue, the method comprising administration to a patient in need thereof an effective amount of a therapeutically effective amount of a compound according to claim 17.

19. (canceled)

20. (canceled)
